(19) United States
(12) Patent Application Publication
(10) Pub. No.: US 2023/0212515 A1
(43) Pub. Date: Jul. 6, 2023

(54) METHOD FOR OBTAINING CAR-NK CELLS

(71) Applicant: GLYCOSTEM THERAPEUTICS B.V., AB Oss (NL)

(72) Inventors: Jan SPANHOLTZ, AB Oss (NL); Nina LAMERS-KOK, AB Oss (NL); Monica RAIMO, AB Oss (NL); Lucia KUCEROVA, AB Oss (NL); Hendrikus Adrianus Maria GEERTS, AB Oss (NL)

(73) Assignee: GLYCOSTEM THERAPEUTICS B.V., AB Oss (NL)

(21) Appl. No.: 17/780,617

(22) PCT Filed: Nov. 29, 2020
(86) PCT No.: PCT/NL2020/050745

§ 371 Date: May 27, 2022

(30) Foreign Application Priority Data
Nov. 28, 2019 (NL) 2024334

(51) Int. Cl. C12N 5/0783; C12N 15/86; A61K 35/17; A61P 35/00; C07K 14/705; C07K 14/725; C07K 16/28

(52) U.S. Cl. CPC C12N 5/0646; C12N 15/86; A61K 35/17; A61P 35/00; C07K 14/70517; C07K 14/70521; C07K 14/70578; C07K 14/7051; C07K 16/2878; C07K 16/2863; C12N 2506/025; C12N 2740/15043; C12N 2501/22; C12N 2501/125; C12N 2501/145; C12N 2501/2306; C12N 2501/2307; C12N 2501/2315; C12N 2501/2302; C12N 2506/11; C12N 2501/26; C07K 2319/02; C07K 2319/03; C07K 2319/33; A61K 38/00

(57) ABSTRACT

The present invention relates to the field of manufacturing of Natural Killer (NK) Cells genetically modified with viral vectors carrying a polynucleotide coding for a Chimeric Antigen Receptors (CARs). The present invention further relates to CAR-NK cells obtained with the method and use of the CAR-NK cells in medicine, in particular for use in a method of treating cancer.

The present invention relates to the field of genetically modified Natural Killer (NK) Cells and methods for manufacturing them. In particular, the present invention relates to CAR-NK cells, methods for manufacturing CAR-NK cells and use of the CAR-NK cells in medicine, in particular for treating cancer.

Natural Killer Cells (NK cells) are innate immune cells with anti-tumour, antiviral and antimicrobial activity. The use of NK cells for the treatment of cancer has attracted interest after successful adoptive transfers and in vivo expansions of NK cells have been reported in patients with cancer [Ruggeri et al (2005) Curr Opin Immunol 17: 211-7; Ren et al (2007) Cancer Biother Radiopharm 22: 223-34; Koehl et al (2004) Blood Cells Mol Dis 33: 261-6.176 Passweg et al (2004) Leukemia 18:1835-8]. In general, donor NK cell infusions were well tolerated without evidence for induction of GvHD in these studies. However, only a few trials investigating adoptive NK cell infusions in patients with cancer have been conducted to date. A major obstacle is that only relatively small numbers of NK cells can be isolated from regular leukapheresis products. This hampers clinical trials for NK-cell dose-dependent anti-tumour responses in humans with cancer [Klingemann et al (2004) Cytotherapy 6: 15-22; Passweg et al (2006) Best Pract Res Clin Haematol 19: 811-824; McKenna et al (2007) Transfusion 47: 520-528; Koehl et al (2005) Klin Padiatr 217: 345-350; Iyengar et al (2003) Cytotherapy 5: 479-484; Meyer-Monard et al (2009) Transfusion 49: 362-371]. Therefore, ex vivo protocols for expansion and activation of NK cells are under investigation enabling clinical trials at higher NK cell dosages and to permit multiple NK cell infusions [Carlens et al (2001) Hum Immunol 62: 1092-1098; Barkholt et al (2009) Immunotherapy 1: 753-764; Berg et al (2009) Cytotherapy 11: 341-355; Fujisaki et al (2009) Cancer Res 69: 4010-4017; Siegler et al. (2010) Cytotherapy 12(6):750-63]. However, most protocols face technical disadvantages by using supportive feeder cell lines that could lead to regulatory problems producing NK cell products for large-scale and multi-center trials. Previously, we have described an alternative cytokine-based culture method with the capability of generating clinically relevant NK cell products with high cell numbers, high purity and functionality from CD34+ hematopoietic stem cells [Spanholtz et al (2010) PLoS One 5: e9221]. We have further optimized the enrichment of CD34+ stem cells and developed a scalable procedure that results in high yields of activated CD34+ stem cell-derived NK cells.

Chimeric Antigen Receptors (CARs) are hybrid molecules comprising three essential units: (1) an extracellular antigen-binding motif, (2) linking/transmembrane motifs, and (3) intracellular T-cell signalling motifs (Long et al (2013) Oncoimmunology 2 (4):e23621).

The antigen-binding motif of a CAR is in general comparable to a single chain Fragment variable (scFv), the minimal binding domain of an immunoglobulin (Ig) molecule. Alternate antigen-binding motifs, such as receptor ligands, intact immune receptors, library-derived peptides, and innate immune system effector molecules (such as NKG2D) also have been engineered. Alternate cell targets for CAR expression (such as NK or gamma-delta T cells) are also under development (Brown et al (2012) Clin Cancer Res 18(8):2199-209; Lehner et al (2012) PLoS One 7 (2):e31210).

While it appears that CARs can trigger NK-cell activation in a manner similar to CAR T cells, a major impediment to the clinical application of this technology to date has been limited by the in vivo expansion of CAR NK-cells, rapid disappearance of the cells after infusion, and disappointing clinical activity. Accordingly, there is an urgent and long felt need in the art for discovering compositions and methods for treatment of cancer using CAR-NK cells that can exhibit intended therapeutic attributes without at least one of the aforementioned short comings.

The present invention addresses these needs by providing a method for manufacturing CAR-NK cells that allows obtaining highly active CAR-NK cells in sufficient quality and quantity for clinical use. The present invention provides a manufacturing process for highly active, off-the-shelf CAR-NK cells to be used for medical treatment, in particular for treating tumours in patients.

The manufacturing process comprises the following steps:

A first step, aiming at transduction of a cellular population of CD34+ stem cells with a CAR.

A second step, aiming at expansion of the transduced CD34+ stem cells and differentiation of the expanded and transduced CD34+ stem cells into CAR-NK progenitor cells and CAR-NK cells. The inventors have surprisingly found that a particular culture condition during transduction has effects on the nature and the composition on the final differentiated CAR-NK cellular population. Cells obtained when such culture condition was used, are of superior quality than cell obtained using other culture conditions. More specifically, in said first step, CD34+ stem cells are obtained from a biological sample, such as umbilical cord blood, bone marrow or peripheral blood, and a polynucleotide coding for a CAR is introduced, e.g. by viral transduction. Said introduction of the polynucleotide is performed in the presence of a culture medium comprising a collection of cytokines. After said first step, the culture thus contains CAR-CD34+ stem cells, which are further expanded and/or differentiated in a culture medium comprising another collection of cytokines.

In a second step, the cellular population is (further) expanded and differentiated into a cellular population containing CAR-NK cells. The CAR-CD34+ stem cells obtained from said first step are preferably first cultured in a second medium, and in a third medium, both having a collection of cytokines, different from said first culture medium, thereby obtaining a collection of cultured cells containing a plurality of CAR-NK cells or CAR-NK progenitor cells or both.

It was surprisingly found that the conditions applied during said first step, allow obtaining a CAR-NK population having a stronger therapeutic effect than a population obtained using culture media with a different constitution of cytokines. Further, the NK-CAR population obtained by a method according to the invention is superior in its effect against a target cell expressing the CAR-ligand when compared with a population of non-CAR NK cells (natural NK cells) obtained using the same culture conditions but without the introduction of a CAR polynucleotide or with a population of CAR-NK cells containing a CAR with an irrelevant, non-targeting scFv protein.

The present invention thus provides a method for the manufacturing of a population of NK-cells genetically modified with a Chimeric Antigen Receptor (CAR), the method comprising transducing CD34+ stem cells in a culture medium with a specific mix of cytokines. The method of the invention allows obtaining a cellular population in which the resulting CAR-NK cells are superior over those known hitherto.

In a first embodiment, the invention provides a method for the manufacturing of a population of cells, genetically modified with a Chimeric Antigen Receptor (CAR) comprising:

(i) a first step comprising:
a) providing a sample comprising CD34+ hematopoietic stem cells
b) purifying the CD34+ hematopoietic stem cells in said sample,
c) culturing the purified CD34+ hematopoietic stem cells in the presence of culture medium I,
d) transducing the purified CD34+ hematopoietic stem cells with a polynucleotide coding for a CAR, thereby obtaining a cellular population comprising CD34+ stem cells expressing said CAR, and
e) optionally culturing the cellular populations in culture medium I for at least 10 hours, preferably at least 16 hours, more preferably at least 24 hours, more preferably at least 36 hours, more preferably at least 48 hours, more preferably at least 60 hours, most preferably at least 72 hours, wherein culture medium I is a basic culture medium, comprising a collection of cytokines, wherein said collection of cytokines comprises Interleukin-7 and one or more of stem cell factor (SCF), flt-3Ligand (FLT-3L), thrombopoietin (TPO), and two or more of granulocyte-macrophage-colony-stimulating factor (GM-CSF), granulocyte-colony-stimulating factor (G-CSF), and interleukin-6 (IL-6).

In step e), the cells are cultured in medium I without addition of a vector comprising said polynucleotide, whereas in step d) vector is added in regular intervals, preferably every 12-36 hours, more preferably every 16-32 hours, more preferably every 20-28 hours, most preferably very 22-26 hours. Preferably, the cells are washed at least once between step d) and step e) in order to dispose of free viral vectors.

In one preferred embodiment, the polynucleotide used for transduction and expression of the CAR does not encode for a CAR that is specific for an antigen expressed on the cell surface of hematopoietic stem cells, NK progenitor cells, or NK cells, in particular on the cell surface of such cells, present in a culture obtained by performing a method according to the invention. Examples of antigens present on such cells, are: CD34, CD56, CD44v6, CD94, NKG2A, NKG2D, CD16, KIRs, CD38, CD123, CD33, and others.

In other words, said polynucleotide encodes for a CAR that is specific for an antigen that is not expressed on the cell surface of a hematopoietic stem cell, NK progenitor cell or NK cell, in particular such antigen is not expressed on the cell surface of any of such cell present in a culture during the method according to the invention.

Typical examples of such antigens, not present on such cells and thus preferred, are: CD3, CD19, EGFR, HSP70, OGD2, CD20, and others.

For culturing and transducing a total of three different media are used:

Culture medium I is a basic culture medium, comprising a collection of cytokines, wherein said collection of cytokines comprises interleukin-7 (IL-7) and one or more of stem cell factor (SCF), flt-3Ligand (FLT-3L), thrombopoietin (TPO), and two or more of granulocyte-macrophage-colony-stimulating factor (GM-CSF), granulocyte-colony-stimulating factor (G-CSF), and interleukin-6 (IL-6). Preferably, the collection of cytokines comprises IL-7 and two or more of SCF, FLT-3L, and TPO, more preferably, the collection of cytokines comprises SCF, FLT-3L, TPO and IL-7. In a preferred embodiment, the collection of cytokines comprises GM-CSF, G-CSF, and IL-6. It is particularly preferred that the culture medium I comprises SCF, FLT-3L, TPO, IL-7, GM-CSF, G-CSF, and IL-6.

Culture medium II is a basic culture medium comprising a collection of cytokines, wherein said collection of cytokines comprises two or more of SCF, FLT-3L interleukin-15 (IL-15) and IL-7 and two or more of GM-CSF, G-CSF, and IL-6. Preferably the collection of cytokines comprises three or more of SCF, FLT-3L, IL-15, and IL-7, more preferably the collection of cytokines comprises SCF, FLT-3L, IL-15 and IL-7. In a preferred embodiment the collection of cytokines comprises GM-CSF, G-CSF, and IL-6. It is particularly preferred that the culture medium II comprises SCF, FLT-3L, IL-15, IL-7, GM-CSF, G-CSF, and IL-6.

Culture medium III is a basic culture medium comprising a collection of cytokines, wherein said collection of cytokines comprises two or more of SCF, IL-7, IL-15 and interleukin-2 (IL-2) and two or more of GM-CSF, G-CSF, and IL-6. Preferably the collection of cytokines comprises three or more of SCF, IL-7, IL-15, and IL-2, more preferably the collection of cytokines comprises SCF, IL-7, IL-15 and IL-2. In a preferred embodiment the collection of cytokines comprises GM-CSF, G-CSF, and IL-6. It is particularly preferred that the culture medium III comprises SCF, IL-7, IL-15, IL-2, GM-CSF, G-CSF, and IL-6.

First, the method of the invention provides the conditions to produce a cellular population containing CD34+ stem cells carrying at least one polynucleotide coding for a CAR. The cellular population is produced according to the following steps:

The starting material to be used in the method of the present invention is a biological sample containing adult (i.e. postembryonic) stem cells also called somatic stem cells. As used herein the term biological sample means a sample derived from human being. In a preferred embodiment the starting material to be used is the umbilical cord blood.

According to the method of the invention CD34+ stem cells are isolated from the biological sample. Different protocols are known in the art for CD34+ isolation including methods based on immunomagnetic selection or cell sorting. As used herein, immunomagnetic selection refers to the coupling of antibodies to magnetic particles thus enabling separation of the antigenic structures by the use of a magnet.

In a preferred embodiment, the biological sample is first enriched for mononuclear cells using gradient separation or centrifugation techniques and is then subjected to immunomagnetic selection by labelling cells with specific anti-CD34+ antibody conjugated to magnetic beads and purifying CD34+ cells using magnetic columns. In a further preferred embodiment immunomagnetic separation is performed using MidiMACS™ Separator, CliniMACS® Plus Instrument or CliniMACS Prodigy® devices.

During the first phase of the method of the invention isolated CD34+ stem cells are first cultured and then transduced in the presence of a basic medium comprising a cocktail of cytokines and growth factors. The culture time before transduction can be any timeframe from a few seconds to 4 days, or more. Preferably, the time between initiating culture and transduction is between 30 minutes and 48 hours, more preferably between 60 minutes and 36 hours, more preferably between 2 hours and 24 hours, most preferably between 6 and 16 hours. Many basic culture media are known. A selection is given below, but many more may be suitable. Basic media include but are not limited to BEM (Basic Eagle Medium), DMEM (Dulbecco's modified Eagle Medium), Glasgow minimal essential medium, M199 basal medium, HAMs F-10, HAMs F-12, Iscove's DMEM, RPMI, Leibovitz L15, MCDB, McCoy 5A, StemSpan H3000® and StemSpanSFEM®, Stemline I™ and Stemline II™ Glycostem Basal growth medium (GBGM™); X-Vivo10™, X-Vivo15™ and X-Vivo20™ etc. Combinations of these basic media can also be used. Preferably serum-free formulations, such as Stemline I™ and Stemline II™, StemSpan H3000®, StemSpan SFEM® or X-Vivo10™, GBGM, X-Vivo15™ and X-Vivo20™ are used at the time point of initiation of culture with or without the addition of human serum.

The amounts given herein are typically suitable for cultures. The amounts may be adapted for different amounts of cells with which cultures are started. The media used in the various culturing steps according to the invention can be varied in their serum content, preferably together with a different combination of cytokines to provide either an expansion medium or a differentiation medium and or alternatively an expansion+differentiation medium at defined time points according to the invention.

Upon isolation, CD34+ stem cells are seeded in containers such as plates, flasks, cell factories or bags at concentration ranging from 500 to 2×10⁶ cells/ml. In a preferred embodiment cells are seeded in step (i)c) at concentration 1×10⁶ cells/ml. In another preferred embodiment, the cell culture of step c) is initiated at a cell density of between 500 and 10,000 CD34+ cells/ml, more preferably between 1,000 and 8,000 CD34+ cells/ml, more preferably between 2,000 and 6,000 CD34+ cells/ml. The inventors have observed that seeding the cells in lower cell densities than done previously does not only result in an absolute increase of haematopoietic stem or progenitor cells after 12-15 days of culture, but it was surprisingly found that further expansion and differentiation culture methods (as described in the Examples and previously in WO2017077096) led to a further increase in expansion and to an equal or better quality of NK cells than a method wherein the CD34⁺ stem cells were cultured in a more dense concentration. One further advantage of starting with low density cell cultures after the purification step (i)b) is that it enables culturing CD34⁺ haematopoietic stem cells obtained from automatic cell sorters without prior manual handling such as, e.g., centrifugation and resuspension in smaller volumes. This is because the cell concentration after automatic cell sorting is in the range of about 500-10,000 CD34⁺ cells/ml, whereas conventionally about 100,000 CD34⁺ cells/ml or more are initially cultured (Veluchamy et al, Front Immunol 2017, 8: 87; Roeven et al, Stem Cells and Development 2015, 24(24): 2886-2898). This enables further automation of the selection and culturing process, which is beneficial for standardization and obtaining marketing authorization.

In one embodiment the cells are seeded in containers previously coated with fragments of fibronectin, for example the fragment CH-296 (RetroNectin®) or functional derivatives. When coated on the surface of cell containers RetroNectin® significantly enhances viral vector-mediated gene transduction into mammalian cells. In a preferred embodiment, cell containers are coated with RetroNectin®.

Once seeded, the CD34+ stem cells are cultured in a culture medium I. In one preferred embodiment, the culture medium I comprises one or more of the cytokines at the following ranges of concentration: GM-CSF between 2-100 pg/ml, preferably between 5-50 pg/ml, most preferably about 10 pg/ml, G-CSF between 100 and 1000 pg/ml, preferably between 150 and 500 pg/ml, most preferably about 250 pg/ml, SCF between 4 ng/ml and 300 ng/ml, preferably between 10 and 100 ng/ml, most preferably about 25 ng/ml, Flt3-L between 4 ng/ml and 300 ng/ml, preferably between 10 and 100 ng/ml, most preferably about 25 ng/ml, TPO between 4 ng/ml and 100 ng/ml, preferably between 10 and 50 ng/ml, most preferably about 25 ng/ml, IL-6 between 5-500 pg/ml, preferably between 25-100 pg/ml, most preferably, about 50 pg/ml, and/or IL7 between 4 ng/ml and 100 ng/ml, preferably between 10 and 50 ng/ml, most preferably about 25 ng/ml. In a more preferred aspect of the invention, the culture medium includes cytokines at a concentration of about 25 ng/ml. SCF, about 25 ng/ml Flt3-L, about 25 ng/ml TPO, about 250 pg/ml G-CSF, about 10 pg/ml GM-CSF, about 50 pg/ml IL-6, and about 25 ng/ml IL7. With “about” is meant in this context a deviation of about 20%, preferably 10%, more preferably 5%, most preferably 2%. Preferably, culture medium I comprises between 0.5-10% serum, more preferably between 1-5% serum, most preferably about 2% serum.

Preferably, the serum is human serum.

After the step of initiating the culture and, optionally, culturing the cells for at least 10 minutes, preferably at least 1 hour, more preferably at least 2 hours, more preferably at least 16 hours or more, the CD34+ stem cells are genetically modified to express at least one polynucleotide coding for a CAR. The technology used to perform genetic engineering of stem cells is viral transduction. As used herein the terms transduction or viral transduction refer to the introduction of foreign polynucleotide into a cell's genome using a viral vector. The term viral vector is used to refer to a viral particle that mediates nucleic acid transfer. In a preferred aspect of the invention, the viral vector to be used in the transduction is selected from a retroviral vector or a Sendai viral vector. A particular useful retroviral vector is a lentiviral vector. Sendai vector is particular useful as it replicates in cytoplasm, does not have DNA form, and infects with high efficiency without the risk of genome insertion. Lentiviral vector is particularly useful for high-efficiency transduction of dividing and non-dividing cells.

According to a method of the invention, transduction is performed by incubating CD34+ stem cells with a viral vector carrying at least one polynucleotide coding for a CAR, in the presence of a culture medium I. In one embodiment, the incubation is performed by substituting at least half of the culture medium with fresh culture medium I, containing viral vectors carrying a polynucleotide coding for a CAR.

In another embodiment the incubation is performed by re-suspending the CD34+ stem cells in fresh culture medium I containing viral vectors carrying a polynucleotide coding for a CAR, such resuspension is then seeded in a cell container at a concentration ranging from 500 to 10×10⁶ cell/ml, preferably from 1,000-2×10⁶ cell/ml, more preferably from 2000-1×10⁶ cell/ml, most preferably between 5000-5×10⁵ cell/ml.

In another aspect of the invention the cell container may be coated with RetroNectin®.

Viral vectors may be incubated with CD34+ stem cells at different concentration depending on the nature of the vector. Preferably transduction is performed by incubation of viral vectors at Multiplicity of Infection (MOI) ranging from 0.01-100. The MOI is the ratio of the number of viral vector particles to the number of target cells present in a defined space. In a preferred embodiment viral vectors are incubated at MOI between 0.1 and 50, more preferably between 1 and 10. During a run of transduction CD34+ stem cells may be incubated with viral vectors for period of time from 5 to 48 hours, preferably from 10-24 hours, more preferably from 12-20 hours. Transduction phase according to the method of the invention may include one or more transduction runs. In a preferred embodiment, a method according to the invention is provided, wherein step (i)d) and (i)e) are repeated at least once, resulting in at least two transduction runs in which CD34+ stem cells are incubated with a viral vector containing said polynucleotide coding for said CAR.

The transduction results in the production of the cellular population containing CAR-CD34+ stem cells carrying at least one polynucleotide encoding a CAR.

In specific embodiments, we generate specific CARs against BCMA (for the treatment of multiple myeloma) and EGFR (for the treatment of metastatic colorectal cancer, metastatic non-small cell lung cancer and head and neck cancer).

BCMA (source: patent US 2016/0046724 A1)
EVQLVESGGGLVQPGGSLRLSCAVSGFALSNHGMSWVRRAPGKGLEWVS
GIVYSGSTYYAASVKGRFTISRDNSRNTLYLQMNSLRPEDTAIYYCSAH
GGESDVWGQGTTVTVSSASGGGGSGGRASGGGGSDIQLTQSPSSLSASV
GDRVTITCRASQSISSYLNWYQQKPGKAPKLLIYAASSLQSGVPSRFSG
SGSGTDFTLTISSLQPEDFATYYCQQSYSTPYTFGQGTKVEIK
EGFR (source: cetuximab antibody sequence):
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSAGGGGSGGGGSGGGGSDILLTQSPVILSV
SPGERVSFSCRASQSIGTNIHWYQQRTNGSPRLLIKYASESISGIPSRF
SGSGSGTDFTLSINSVESEDIADYYCQQNNNWPTTFGAGTKLELKR

The so obtained cellular population containing CAR-CD34+ stem cells may be cultured for at least one further day in the presence of fresh culture medium I before moving to the second phase of the manufacturing method.

In another embodiment, the cellular population containing CAR-CD34+ stem cells may be frozen at the end of the transduction. In another embodiment, the cellular population may be cultured for at least one further day in the presence of fresh culture medium I and then may be frozen.

The manufacturing can be continued after the end of the transduction or after one or more days of culture, or upon thawing of frozen cellular population containing CAR-CD34+ stem cells. Preferably, second culturing phase is performed after a total of at least 7 culturing days in culture medium I.

In one embodiment the invention provides a cellular population containing CAR-CD34+ stem cells, obtainable by step i of the method of the present invention.

The thus obtained CAR-CD34+ stem cells may be purified by positive selection on the expressed CAR. Positive selection may take place through antibodies directed to the expressed CAR or to a selectable protein, co-expressed with the CAR and not normally expressed by the CD34+ cells. Thus, enabling positively selecting CAR expressing stem cells.

As said before, the culture conditions wherein the transduction takes place (i.e. culturing in culture medium I) are preferably continued for a total of at least 7 days, i.e. including culturing before transduction, culturing during transduction runs 1 and 2, and culturing after transduction. Thereafter, a second phase, comprising expansion and differentiation into NK cells is preferably initiated.

In a preferred embodiment, a method according to the invention is provided, the method further comprising:

(ii) a second step in which the cellular population from step (i) is expanded and differentiated into a cellular population containing CAR-NK cells, the step comprising culturing the cellular population from step (i) containing CAR-CD34+ stem cells in culture medium III, thereby obtaining a cellular population containing CAR-NK cells and progenitor CAR-NK cells wherein the culture medium III is a basic culture medium comprising a collection of cytokines, wherein said collection of cytokines comprises two or more of SCF, IL-7, IL-15 and interleukin-2 (IL-2) and two or more of GM-CSF, G-CSF, and IL-6.

Alternatively, a method according to the invention is provided, the method further comprising:

(ii) a second step in which the cellular population from step (i) is expanded and differentiated into a cellular population containing CAR-NK cells and progenitor CAR-NK cells, the step comprising culturing the CAR-CD34+ stem cells from step (i) in culture medium II, thereby obtaining a cellular population containing CAR stem cells and CAR progenitor cells, and

(iii) a third step in which the cellular population from step (ii) is further expanded and differentiated into a cellular population containing CAR-NK cells and progenitor CAR-NK cells,

wherein culture medium II is a basic culture medium comprising a collection of cytokines, wherein said collection of cytokines comprises two or more of SCF, FLT-3L interleukin-15 (IL-15) and IL-7 and two or more of GM-CSF, G-CSF, and IL6, and

wherein culture medium III is a basic culture medium comprising a collection of cytokines, wherein said collection of cytokines comprises two or more of SCF, IL-7, IL-15 and interleukin-2 (IL-2) and two or more of GM-CSF, G-CSF, and IL-6.

Thus, as can be deducted from the above two paragraphs, the culturing step with medium II is optional and is directed to obtaining more CAR-stem- or CAR-NK-progenitor cells to enter culturing step with medium III, which aims at differentiation.

In one embodiment, the invention thus provides a method for the manufacturing of a population of NK cells and progenitor NK cells, the method comprising:

(i) a first step comprising:
a) providing a sample comprising CD34+ hematopoietic stem cells
b) purifying the CD34+ hematopoietic stem cells in said sample,
c) culturing the purified CD34+ hematopoietic stem cells in the presence of culture medium I,
d) introducing a polynucleotide coding for a CAR, thereby obtaining a cellular population comprising CD34+ stem cells expressing said CAR, and
e) optionally culturing the cellular populations in culture medium I for at least one day, wherein
culture medium I is a basic culture medium, comprising a collection of cytokines, wherein said collection of cytokines comprises Interleukin-7 and one or more of stem cell factor (SCF), flt-3Ligand (FLT-3L), thrombopoietin (TPO), and two or more of granulocyte-macrophage-colony-stimulating factor (GM-CSF), granulocyte-colony-stimulating factor (G-CSF), and interleukin-6 (IL-6).
(ii) an optional second step in which the cellular population from step (i) is expanded and differentiated into a cellular population containing CAR-NK cells and progenitor CAR-NK cells, the step comprising culturing the CAR-CD34+ stem cells from step (i) in culture medium II, thereby obtaining a cellular population containing CAR stem cells and CAR progenitor cells, and
(iii) a third step in which the cellular population from step (i) or from step (ii) is further expanded and differentiated into a cellular population containing CAR-NK cells and progenitor CAR-NK cells,
wherein culture medium II is a basic culture medium comprising a collection of cytokines, wherein said collection of cytokines comprises two or more of SCF, FLT-3L interleukin-15 (IL-15) and IL-7 and two or more of GM-CSF, G-CSF, and IL6, and

wherein culture medium III is a basic culture medium comprising a collection of cytokines, wherein said collection of cytokines comprises two or more of SCF, IL-7, IL-15 and interleukin-2 (IL-2) and two or more of GM-CSF, G-CSF, and IL-6 In the optional step, cells collected from step (i) are cultured at a cell density of at least 0.5×10⁶ ml for at least 4 days in culture medium II thereby obtaining a collection of cultured CAR, stem cells, CAR, progenitor cells or both, containing a plurality of CAR progenitor cells committed to the NK cell lineage.

During the second culturing phase, the cellular population containing CAR-CD34+ stem cells obtained after the culturing step with medium I, or the cellular population containing CAR-stem- or CAR-NK-progenitor cells obtained after the culturing step with medium II is cultured for at least 7 days at a cell density of at least 1×10⁶/ml in culture medium III, thereby obtaining a collection of cultured cells containing a plurality of CAR-NK cells or CAR-NK progenitor cells or both.

In one preferred embodiment the culture medium II comprises one or more of the cytokines at the following ranges of concentration: G-CSF between 50 pg/ml and 1000 ng/ml, preferably between 150 and 400 ng/ml, GM-CSF between 2-100 pg/ml, preferably between 5-25 ng/ml, SCF between 4-300 ng/ml, preferably between 10-100 ng/ml, Flt3-L between 4 ng/ml and 300 ng/ml, preferably between 10 and 100 ng/ml, IL15 between 4 ng/ml and 300 ng/ml, preferably between 10 and 100 ng/ml, IL-6 between 2 and 500 pg/ml, preferably between 20 and 200 pg/ml and/or IL7 between 4 ng/ml and 100 ng/ml, preferably between 10 and 50 ng/ml. In a more preferred aspect of the invention, the culture medium II includes cytokines at a concentration of about 10 pg/ml GM-CSF, about 250 pg/ml G-CSF, about 25 ng/ml. SCF, about 25 ng/ml Flt3-L, about 20 ng/ml IL-15, about 50 pg/ml IL-6, and about 25 ng/ml IL7. With “about” is meant in this context a deviation of about 20%, preferably 10%, more preferably 5%, most preferably 2%. Preferably, culture medium II comprises between 4-20% serum, more preferably between 6-15% serum, most preferably about 10% serum. Preferably, the serum is human serum.

In one preferred embodiment the culture medium III comprises one or more of the cytokines at the following ranges of concentration: G-CSF between 50 pg/ml and 1000 ng/ml, preferably between 150 and 400 ng/ml, GM-CSF between 2-100 pg/ml, preferably between 5-25 ng/ml, SCF between 4-300 ng/ml, preferably between 10-100 ng/ml, IL-2 between 200 U/ml and 5000 U/ml, preferably between 500 and 2000 U/ml, IL15 between 4 ng/ml and 300 ng/ml, preferably between 10 and 100 ng/ml, IL-6 between 2 and 500 pg/ml, preferably between 20 and 200 pg/ml and/or IL7 between 4 ng/ml and 100 ng/ml, preferably between 10 and 50 ng/ml. In a more preferred aspect of the invention, the culture medium III includes cytokines at a concentration of about 10 pg/ml GM-CSF, about 250 pg/ml G-CSF, about 20 ng/ml. SCF, about 20 ng/ml IL-15, about 50 pg/ml IL-6, about 1000 U/ml IL-2, and about 20 ng/ml IL7. With “about” is meant in this context a deviation of about 20%, preferably 10%, more preferably 5%, most preferably 2%. Preferably, culture medium II comprises between 4-100 μg/ml heparin, preferably between 10-40 μg/ml heparin, more preferably about 20 μg/ml heparin. Preferably, culture medium III comprises between 0.5-10% serum, more preferably between 1-5% serum, most preferably about 2% serum. Preferably, the serum is human serum.

Chimeric Antigen Receptors

Viral vectors to be used in the method of the invention carry at least one polynucleotide coding for a CAR. The CARs to be used in the method of the present invention are recombinant chimeric receptors comprising:
(i) An extracellular part: This part consists of a signal peptide responsible for the secretion of the CAR to the exterior of the cell membrane. The signal sequence is immediately followed by the targeting or antigen-specific binding domain. This domain consists of a polypeptide sequence that binds an antigen with high specificity. This domain can consist of any kind of antibody (monoclonal, polyclonal, single or multiple chain) with high specificity. Most commonly, single chain variable fragments (scFvs) are used to deliver CAR-specificity. The targeting domain is followed by a hinge region, which gives the CAR its flexibility, and a spacer domain, usually the constant region of the IgG1 heavy chain.
(ii) A transmembrane part: This part anchors the CAR into the NK-cell membrane
(iii) A cytoplasmic part: This part contains the various activating and co-stimulatory domains. These domains transmit the signal upon binding of the scFv to its target to the intracellular signalling pathways.

The term tumour antigen includes antigens expressed on a tumour cell, such as, but not limited to biomarkers or cell surface markers that are found on tumour cells and are substantially absent on normal tissues, or restricted in their expression to nonvital normal tissues. With “substantially absent” is meant that the expression level on at least the vital normal cells is so low that the CAR-NK cell shows relatively little binding to said normal cell and, thus toxicity is low.

The invention further provides a composition comprising CAR-NK cells obtainable by the method of the present invention. It was surprisingly found that condition applied for transduction of stem cells with viral vectors carrying a Chimeric Antigen Receptors has impact on the nature and composition of the cellular population containing CAR-CD34+ stem cells as well as on the final cellular population containing CAR-NK cells, resulting from the expansion and differentiation phase. CAR-NK cells obtainable according to the method of the invention present a synergistic therapeutic effect between the NK-cells and the CAR. Such effect results to be stronger than that obtained with a different manufacturing method. Therefore, the invention further provides a composition comprising CAR-NK cells obtainable by the method of the present invention for use in medicine, more preferably for use in immunotherapy, in particular for the treatment of tumours and haematological malignancies.

The term “immunotherapy” denotes a treatment that uses certain parts of a person's immune system to fight diseases such as cancer. The parts of the immune system can be either from the person having the disease, but also from another person, called “donor”, such as the case in the present invention. A composition for use according to the invention is preferably used in cell-based immunotherapy, wherein immune effector cells, derived from an autologous, non-haploidentical donor are administered to a recipient in need thereof.

This invention preferably uses cells that are generated with a GMP-compliant culture system for the generation of large batches of immune effector cells, e.g. from umbilical cord blood (UCB)-derived CD34+ progenitor cells, preferably without T cell contamination. It is advantageous to use such cells as they have higher conformity, making, e.g., regulatory processes much easier. At the same time, the present invention enables usage of such large batches of immune effector cells, because previously, individual batches had to be generated, based on the at least partial match with the envisaged recipient because of safety concerns. The present invention, however, shows that immune effector cells as defined by the invention, mismatched beyond being haploidentical are safe to use in immunotherapy and that they show efficacy.

Preferably, a composition for use according to the invention further comprises at least one excipient, such as for instance water for infusion, physiologic salt solution (0.9% NaCl), or a cell buffer, preferably consisting of a physiologic salt solution substituted with a protein component such as human serum albumin (HAS).

In order for a composition of the invention to be used, e.g. in non-haploidentical mismatched situation, it is preferred that the composition for use according to the invention is low on B-cell or a T-cell numbers to avoid graft versus host disease. In a preferred embodiment, a composition for use according to the invention does not result in graft versus host disease. Preferably, the composition comprises at not more than 5% T cells and not more than 5% B cells, more preferably not more than 2% T cells and not more than 2% B cells, most preferably less than 1% T cells and less than 1% B cells.

In a preferred embodiment, a composition for use according to the invention is provided, wherein the immune effector cell is, next to being positive for a CAR, positive for Neural Cell Adhesion Molecule (NCAM).

Neural cell adhesion molecule (NCAM), is a glycoprotein of Immunoglobulin (Ig) superfamily expressed on the surface of neurons, glia, skeletal muscle and natural killer cells. NCAM, also called CD56, has been implicated as having a role in cell-cell adhesion, neurite outgrowth, synaptic plasticity, and learning and memory. NCAM is preferably used to define the population of differentiated immune effector cells for use according to the invention and can be used to discriminate the infused effector cells from patient's natural killer cells in the peripheral blood. Preferably, the composition for use according to the invention comprises more than 90% CD56+ cells, more preferably more than 95% CD56+ cells, most preferably more than 98% CD56+ cells.

Typically, the composition of the invention comprises a plurality of cells. It is not necessary for all the cells in the composition to have the features and effects as defined by the invention. However, it is preferred to have at least a certain percentage of immune effector cells as defined in the invention in the composition for use according to the invention in order to have the right balance with regard to efficiency (during production) and efficacy (in the clinics). In a preferred embodiment, a composition for use according to invention is provided, wherein the composition comprises a plurality of cells, characterized in that 30-100%, preferably 30-90%, more preferably 30-80%, more preferably 30-70%, more preferably 30-60%, more preferably 30-50%, most preferably 30-40% of the plurality of cells is CAR-NK cell as defined by the invention. Preferably, the composition comprising a plurality of cells is characterized in that 40-100%, more preferably 50-100%, more preferably 60-100%, more preferably 70-100%, more preferably 80-100%, most preferably 90-100% of the plurality of cells is a CAR-NK cell as defined by the invention. Other preferred ranges of CAR-NK cells as defined by the invention within a composition for use according to the invention are: 40-90%, 50-90%, 60-90%, 70-90%, 80-90%, 40-80%, 50-80%, 60-80%, 40-70%, 40-60%, 50-60% or 40-50%. For production efficiency, a lower percentage of the CAR-NK cells as defined by the invention is desired, whereas on the other hand for clinical efficacy and for regulatory reasons a higher percentage of the CAR-NK cells as defined by the invention is desired.

It is preferred, from a regulatory perspective, but also from a perspective of efficiency, that a composition for use according to the invention is obtained from a single donor. Even more preferred is that a single donor provides more than one treatment dose, such that large scale batches can be produced, be cleared or certified, and used off-the-shelf at the moment a random individual must be treated with a composition for use according to the invention. Preferably the generation of CAR-NK cells suffices for at least 10, more preferably at least 20, more preferably at least 50, more preferably at least 100, most preferably at least 200 or more single treatment doses for use according to the invention. If e.g. about 5×10⁸-1×10¹⁰ cells are to be used for a single treatment, it is preferred that for treating, e.g. 10 individuals at least 10¹¹ immune effector cells are generated from the CD34 positive stem or progenitor cells from one single donor. The thus generated large batch of cell can be easily transferred to vials or bags with the correct amount of cells (e.g. about 5×10⁸-1×10¹⁰) cells per vial or bag, frozen and stored. In the moment a composition for use according to the invention is needed, one of such vials or one of such bags can be thawed and prepared for administration to the individual in need of immunotherapy. In a preferred embodiment, a composition for a use according to invention is provided, wherein the plurality of cells is derived from cells obtained from a single donor. Preferably, the plurality of cells is derived from at least one of umbilical cord blood and bone marrow, as these are rich sources of CD34 positive stem and/or progenitor cells.

Because of the possibility to use off-the-shelf compositions comprising CAR-NK cells the composition for use according to the invention shifts cell adoptive therapy a step further from personalized medicine towards more generic medication as it is no longer necessary to search for individual donors to match individual recipient. This also has a beneficial impact on the costs of treatment.

With “off-the-shelf” as used herein is meant that such composition is prepared and stored for direct usage when needed. In particular, a composition that is available “off-the-shelf” is not generated for one specific recipient but in general can be used for different recipients at different time points. The composition as defined by the invention can for instance be frozen and, when needed, thawed and used as defined by the invention. A composition as defined by the invention enables large scale production of GMP generated immune effector cells that can theoretically be provided within minutes when needed for any random recipient.

In one embodiment, the invention provides a composition comprising a CAR-NK cell, wherein the composition is generated ex vivo in a process comprising the steps of:

(i) A first step comprising:
a) providing a sample comprising CD34+ hematopoietic stem cells
b) purifying the CD34+ hematopoietic stem cells in said sample,
c) culturing the purified CD34+ hematopoietic stem cells in the presence of culture medium I,
d) introducing a polynucleotide coding for a CAR, thereby obtaining a cellular population comprising CD34+ stem cells expressing said CAR, and
e) optionally culturing the cellular populations in culture medium I for at least one day, wherein
culture medium I is a basic culture medium, comprising a collection of cytokines, wherein said collection of cytokines comprises Interleukin-7 and one or more of stem cell factor (SCF), flt-3Ligand (FLT-3L), thrombopoietin (TPO), and two or more of granulocyte-macrophage-colony-stimulating factor (GM-CSF), granulocyte-colony-stimulating factor (G-CSF), and interleukin-6 (IL-6).
(ii) an optional second step in which the cellular population from step (i) is expanded and differentiated into a cellular population containing CAR-NK cells and progenitor CAR-NK cells, the step comprising culturing the CAR-CD34+ stem cells from step (i) in culture medium II, thereby obtaining a cellular population containing CAR stem cells and CAR progenitor cells, and
(iii) a third step in which the cellular population from step (i) or from step (ii) is further expanded and differentiated into a cellular population containing CAR-NK cells and progenitor CAR-NK cells, wherein culture medium II is a basic culture medium comprising a collection of cytokines, wherein said collection of cytokines comprises two or more of SCF, FLT-3L interleukin-15 (IL-15) and IL-7 and two or more of GM-CSF, G-CSF, and IL6, and
wherein culture medium III is a basic culture medium comprising a collection of cytokines, wherein said collection of cytokines comprises two or more of SCF, IL-7, IL-15 and interleukin-2 (IL-2) and two or more of GM-CSF, G-CSF, and IL-6.

A sample comprising hematopoietic stem and/or progenitor cells may be obtained in any possible way, such as for instance obtain or collect a stem and/or progenitor containing cell source, such as bone marrow, cord blood, placental material, peripheral blood, peripheral blood of a person treated with stem cell mobilizing agents, generated ex vivo from embryonic stem cells or any deviates thereof using cell culturing steps or generated ex vivo from induced pluripotent stem cells and any deviates thereof using cell culturing steps. Hematopoietic stem and/or progenitor cells can be further purified from such stem and/or progenitor containing cell sources using affinity purification methods.

With the term “ex vivo” is meant that the process or method performed is not used within a living individual, but for instance in a device able to culture cells, preferably an open or a closed cell culture device, such as a culture flask, a disposable bag or a bioreactor.

With the term “CD34+ stem cell” is meant a multipotent stem cell, which expresses the CD34 antigen on the cell surface, preferably being a stem cell, which is able to develop in all certain types of blood cells and more preferably a cell, which can give rise to lineage specific progenitor cells of the blood lineages.

With the term “CD34+ progenitor cell” is meant a multipotent progenitor cell, which expresses the CD34 antigen on the cell surface, preferably being a progenitor cell, which is able to develop in various types of blood cells and more preferably a cell, which can give rise to lineage specific progenitor cells of the certain blood lineages.

With the term “affinity purification” as used herein is meant, that the cells to be purified are labelled, by targeting for instance a specific epitope of interest for separation purposes, for instance targeting an antigen with an antibody coupled to an agent suitable for detection by a method for separation, using for instance antibodies coupled to fluorochromes for purification methods such as fluorescence activated cell sorting (FACS), and/or using for instance antibodies coupled to magnetic particles for magnetic selection procedures. Affinity purification methods are known in the art and can for instance be any method of separating biochemical mixtures based on a highly specific interaction such as that between antigen and antibody, enzyme and substrate, or receptor and ligand.

With the term “expanding” as used herein is meant multiplication of cells due to cell division events caused by a cell culturing step, preferably without essentially changing the phenotype of the cell, which is generally called “differentiation”. With the phrase “without essentially changing the phenotype of the cell” is meant that the cell preferably does not change its function, its cell surface markers and/or its morphology.

With the term “differentiating” as used herein is meant changing the phenotype of the cell, which means changing the expression of certain surface molecules during the cell culture process, changing the cells function and/or changing the morphology of the cell, wherein the cell preferably still can expand due to the addition of cell culture medium. As indicated previously, the inventors have shown that a composition for use in immunotherapy as defined by the invention is particularly useful for the treatment of a tumour. According to a preferred embodiment, the composition for use according to the invention is for the treatment of a tumour. Tumour, within the meaning of the invention, includes hematopoietic tumours or solid tumours. The tumour can either be malign or benign.

A composition for use in immunotherapy according to the invention can be used at different stages in the treatment of tumours, in particular in the treatment of hematopoietic tumours, such as e.g. acute myelogenous leukaemia (AML). For instance, as exemplified by the current invention, the composition can preferably be used as consolidation therapy in those (elderly) patients not eligible to undergo a bone marrow transplant. Additionally, as shown by others using another treatment, immune effector cell therapy according to the invention can preferably be used for patients not reaching complete remission on induction therapy (refractory patients), or those relapsing shortly after induction therapy (recurrent patients). Incorporation of immune effector cell therapy into other consolidation therapies is also feasible and preferred, such as the additional use of immune effector cells as defined by the invention in allogeneic HSTC regimens.

In a preferred embodiment, a composition for a use according to the invention is provided, wherein the composition to be administered in one treatment comprises at least 5×10⁵ cells and not more than 5×10¹¹ cells.

The composition of the invention can be administered through any acceptable method, provided the immune effector cells are able to reach their target in the individual. It is for instance possible to administer the composition of the invention via the intravenous route or via a topical route, including but not limited to the ocular, dermal, pulmonary, buccal and intranasal route. With topical route, as used herein, is also meant any direct local administration such as for instance in the bone marrow, but also directly injected in, e.g., a solid tumour. In particular cases, e.g. if the immunotherapy is aimed at an effect on the mucosal layer of the gastrointestinal tract, the oral route can be used.

Preferably, a composition for a use according to the invention is provided, wherein the composition is administered by intravenous route or by a topical route or by oral route or by any combination of the three routes. With the term “topical” as used herein is meant, that the immune effector cells are applied locally, preferably at the site of tumour, which can be localized in any anatomical site, more specifically the tumour can be localized in the bone marrow or any other organ. The composition for use according to the invention can be administered once, but if deemed necessary, the composition may be administered multiple times. These can be multiple times a day, a week or even a month. It is also possible to first await the clinical result of a first administration, e.g. an infusion and, if deemed necessary, give a second administration if the composition is not effective, and even a third, a fourth, and so on.

In one preferred embodiment, a composition for a use according to the invention for the treatment of a tumour is provided, wherein the tumour is a hematopoietic or lymphoid tumour or wherein tumour is a solid tumour.

With the term “haematological”, “haematopoietic” or “lymphoid” tumour is meant, that these are tumours of the hematopoietic and lymphoid tissues.

Hematopoietic and lymphoid malignancies are tumours that affect the blood, bone marrow, lymph, and lymphatic system.

In those cases that the tumour is a haematopoietic or lymphoid tumour, a composition for use according to the invention is provided, wherein the tumour is one or more of leukaemia, lymphoma, myelodysplastic syndrome or myeloma, preferably a leukaemia, lymphoma or myeloma selected from acute myelogenous leukaemia (AML), chronic myelogenous leukaemia (CML), acute T cell leukaemia, acute lymphoblastic leukaemia (ALL), chronic lymphocytic leukaemia (CLL), acute monocytic leukaemia (AMoL), mantle cells lymphoma (MCL), histiocytic lymphoma or multiple myeloma, preferably AML.

In those cases that the tumour is a solid tumour, a composition for use according to the invention is provided, wherein the tumour is one of malignant neoplasms or metastatic induced secondary tumours of adenocarcinoma, squamous cell carcinoma, adenosquamous carcinoma anaplastic carcinoma, large cell carcinoma or small cell carcinoma, hepatocellular carcinoma, hepatoblastoma, colon adenocarcinoma, renal cell carcinoma, renal cell adenocarcinoma, colorectal carcinoma, colorectal adenocarcinoma, glioblastoma, glioma, head and neck cancer, lung cancer, breast cancer, Merkel cell cancer, rhabdomyosarcoma, malignant melanoma, epidermoid carcinoma, lung carcinoma, renal carcinoma, kidney adenocarcinoma, breast carcinoma, breast adenocarcinoma, breast ductal carcinoma, non-small cell lung cancer, ovarian cancer, oral cancer, anal cancer, skin cancer, Ewing sarcoma, stomach cancer, urethral cancer, uterine cancer, uterine sarcoma, vaginal cancer, vulvar cancer, Wilms tumour, Waldenström macroglobulinemia, pancreas carcinoma, pancreas adenocarcinoma, cervix carcinoma, squamous cell carcinoma, medulloblastoma, prostate carcinoma, colon carcinoma, colon adenocarcinoma, transitional cell carcinoma, osteosarcoma, ductal carcinoma, large cell lung carcinoma, small cell lung carcinoma, ovary adenocarcinoma, ovary teratocarcinoma, bladder papilloma, neuroblastoma, glioblastoma multiforma, glioblastoma astrocytoma, epithelioid carcinoma, melanoma or retinoblastoma.

In a preferred embodiment, a composition for use according to the invention is provided, wherein the solid tumour is selected from malignant neoplasms or metastatic induced secondary tumours of cervical cancers selected from adenocarcinoma, squamous cell carcinoma, adenosquamous carcinoma, cervix carcinoma, small cell carcinoma, and melanoma. In another preferred embodiment, a composition for use according to the invention is provided, wherein the solid tumour is selected from malignant neoplasms or metastatic induced secondary tumours of colorectal cancers selected from adenocarcinoma, squamous cell carcinoma, colon adenocarcinoma, colorectal carcinoma, colorectal adenocarcinoma, colon carcinoma, and melanoma.

The composition of the invention has several advantages with respect to treatment options known to date. The composition of the invention is beneficial independent of HPV types, tumour histology, tumour EGFR expression and KRAS status. In addition to it, the immune effector cell of the invention also overcomes HLA-E, HLA-G and (IDO) inhibition, thus resulting in enhanced anti-tumour effects against tumours, especially against cervical cancers and colorectal cancers.

The term “Epidermal growth factor receptor” or EGFR as it is commonly described, refers to a cell surface protein widely expressed in almost all healthy tissues. The EGFR protein is encoded by transmembrane glycoprotein and is a member of the protein kinase family. Overexpression of EGFR and mutations in its downstream signalling pathway has been associated with bad prognosis in several solid tumours like colon, lung and cervix.

The term Kirsten rat sarcoma viral oncogene (KRAS) refers to the gene actively involved in regulating normal tissue signalling, part of EGFR downstream signalling pathway. However, mutations in the KRAS gene has been reported in tumour cells in solid tumours of colon, rectum and lungs. These activating mutations occurring in more than 50% of colorectal cancer patient helps tumour cells to evade EGFR targeting drugs like cetuximab and panitumumab.

The term “human papilloma virus (HPV) as used herein refers to the group of viruses which causes cervical cancer in women. HPV virus affects the skin and moist membranes surrounding mouth, throat, vulva, cervix and vagina. HPV infection causes abnormal cell changes that leads to cancer in the cervix.

The term Indoleamine 2,3 dioxygenase (IDO) as used herein refers to an enzyme which acts as a catalyst in degrading amino acids L-tryptophan to N-formylkynurenine. Overexpression of IDO commonly reported in solid tumours of prostate, gastric, ovarian, cervix and colon, enables tumour cells to evade killing by cytotoxic T cells and NK cells.

The invention is described in more detail in the following, non-limiting examples.

### DESCRIPTION OF THE DRAWINGS

FIG. 1: We designed a modular CAR molecule that allows easy incorporation of novel scFv proteins via a single cloning procedure. The FseI-SbfI-flanked stuffer fragment allows easy seamless cloning of targeting molecules into the empty CAR backbone. As shown in FIGS. 1b and c, we inserted scFv proteins into this space which target the transduced NK cell to respectively BCMA+ or EGFR+ tumor cells. FIG. 1d illustrates a control vector that contains an irrelevant scFv that has no binding affinity to any human protein. The IgD hinge region confers the flexibility to the CAR, while the optimized IgG1 heavy chain sequence functions as spacer region. The CAR is anchored into the membrane via de CD28 transmembrane sequence. This 3^{rd} generation CAR contains the co-stimulatory domains of CD28 and OX-40 and the activation domain of CD3zeta. FIG. 1e shows the location of each domain with respect to the cell membrane. Arrows indicate unique restriction sites that can be used to incorporate scFvs (via FseI and SbfI), transfer the CAR into a lentiviral transfer plasmid (arrows on either side of the CAR) or to change co-stimulatory or activation domains (arrows flanking each domain)

FIG. 2: Representative example of flow cytometry data from two different culture conditions using two donors. Viable cells are gated on CD45+ lymphocytes. The figure illustrates the expression of CD44v6 on NK cells (A) and progenitor cells (B) if hematopoietic stem and progenitor cells are expanded and differentiated according to technology described in this invention. A. CD44v6 expression of day 29 oNKord cells (% CD56: 92%), B. CD44v6 expression on day 14 oNKord progenitor cells (% CD56: 1.2%)

FIG. 3: Transduction efficiency in CD34+ cells. CD34+ HSC cells were transduced with VSV-G pseudotyped lentiviral particles containing EGFP at MOI 20. Cells were expanded in expansion/differentiation medium for subsequent 29 days and the percentage of EGFP positive cells was determined by flow cytometry. Three different donors were used for the transduction, measurements were performed in triplicates, mean±SD is shown.

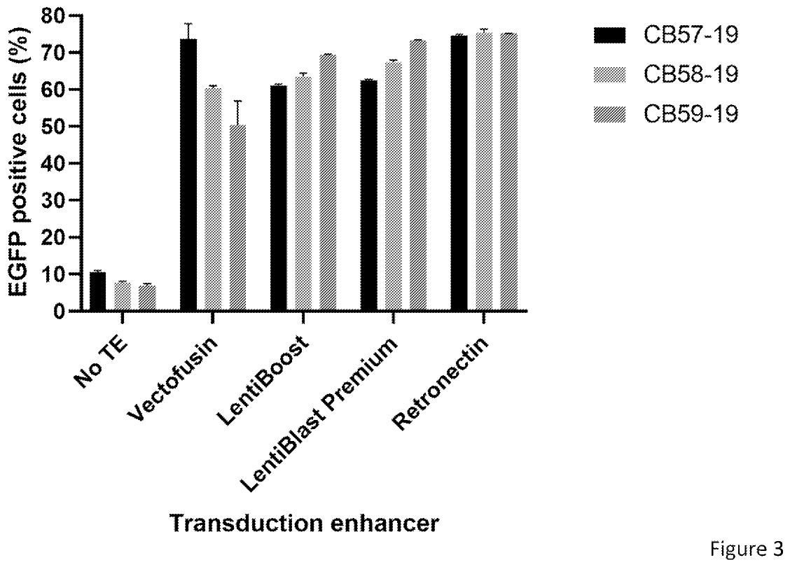

FIG. 4: Transduction of precursors derived from CD34+ HSC. Precursor cells derived from CD34+ HSC (NK) were transduced with VSV-G pseudotyped lentiviral particles containing EGFP at MOI 20 after 21, 28 and 34 days in culture in expansion/differentiation medium. Cells were maintained in differentiation medium for subsequent 6 days after transduction and the percentage of EGFP positive cells was determined by flow cytometry. Three different donors were used for the transduction, measurements were performed in duplicates, mean of three different donors±SD is shown.

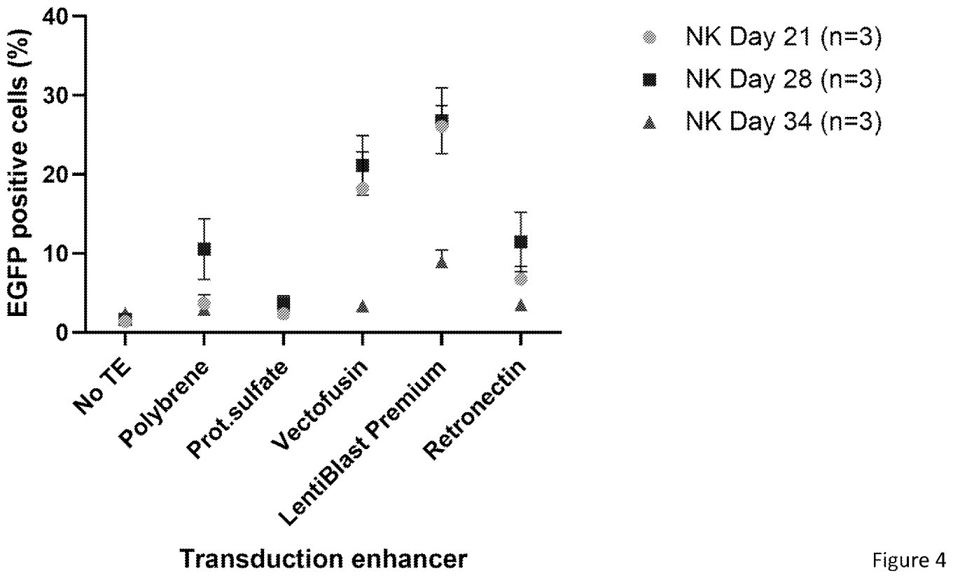

### EXAMPLES

### Example 1: Generation of a 3^{rd} Generation CAR Construct

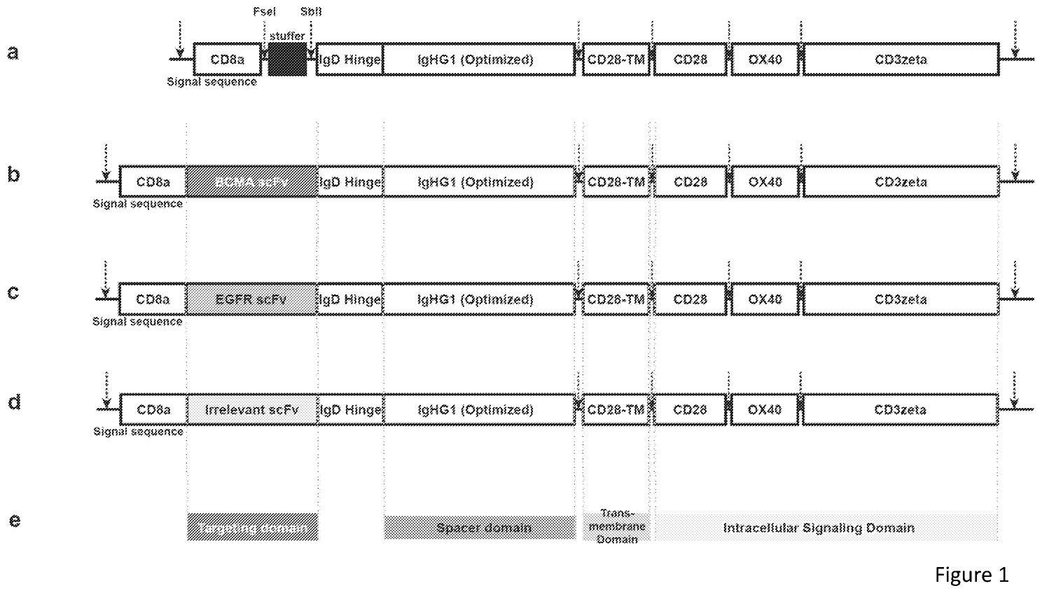

The CAR construct used in these examples is synthetically generated by ID&T DNA technologies as a single polynucleotide with flanking restriction sites that allow easy transfer into a suitable expression vector. The CAR expression cassette is fully human codon optimized for efficient expression in human cells. The vector consists of a CD8a signal peptide, an FseI-SbfI flanked stuffer fragment, the IgD hinge region, the IgG1 heavy constant fragment optimized to avoid binding to IgG Fc gamma receptors and thus to inhibit unintentional activation of innate immune cells (Hombach et al., Gene Therapy, 2010), the CD28 transmembrane domain, the CD28 co-activation domain, the OX40 co-activation domain and the CD3zeta activation domain. Each (co-) activation domain is flanked by unique restriction sites that allows testing of each individual domain.
A schematic version of this CAR cassette is shown in FIG. 1.
The protein sequence of this CAR cassette is shown below:

CD8α leader sequence:
MDALPVTALLLPLALLLHAEVQLQQS
FseI-SbfI flanked staffer fragment:
GRPSLSESCRA
IgD hinge region:
AFRWPESPKAQASSVPTAQPQAEGSL
Optimized IgG1 heavy chain Fc part, optimized:
AKATTAPATTRNTGGRGGEEKKKEKEKEEQEERETKTPEAAAVEPKSCD
KTHTCPPCAAPPVAGPSVFLFPPKPKDTLMIARTPEVTCVVVDVSHEDP
EVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQ
QGNVFSCSVMHEALHNHYTQKSLSLSPGK
CD28 transmembrane domain:
FWVLVVVGGVLACYSLLVTVAFIIFWV
CD28 co-activation domain:
RSKRSRLLHSDYMNMTPRRPGPTRKHYQPYAPPRDFAAYRS
OX40 co-activation domain:
RRDQRLPPDAHKPPGGGSFRTPIQEEQADAHSTLAKI
CD8zeta activation domain:
RVKFSRSAEPPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

To generate specific CARs against BCMA (for the treatment of multiple myeloma) and EGFR (for the treatment of metastatic colorectal cancer, metastatic non-small cell lung cancer and head and neck cancer), we substitute the stuffer fragment for single-chain variable fragment protein sequences against BCMA and EGFR by seamless cloning.

BCMA (source: patent US 2016/0046724 A1):
EVQLVESGGGLVQPGGSLRLSCAVSGFALSNHGMSWVRRAPGKGLEWVS
GIVYSGSTYYAASVKGRFTISRDNSRNTLYLQMNSLRPEDTAIYYCSAH
GGESDVWGQGTTVTVSSASGGGGSGGRASGGGGSDIQLTQSPSSLSASV
GDRVTITCRASQSISSYLNWYQQKPGKAPKLLIYAASSLQSGVPSRFSG
SGSGTDFTLTISSLQPEDFATYYCQQSYSTPYTFGQGTKVEIK
EGFR (source: cetuximab antibody sequence):
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSAGGGGSGGGGSGGGGSDILLTQSPVILSV
SPGERVSFSCRASQSIGTNIHWYQQRTNGSPRLLIKYASESISGIPSRF
SGSGSGTDFTLSINSVESEDIADYYCQQNNNWPTTFGAGTKLELKR

The entire CAR cassette (including relevant ScFv sequence) is inserted into a 3^{rd} generation lentiviral backbone plasmid via unique restriction sites flanking the CAR cassette. In this vector, the EF1alpha promoter drives the constitutive expression of the CAR polynucleotide.
Viral vectors are produced in HEK293T cells by transfection of 3^{rd} generation helper plasmids in combination with the selected lentiviral transfer plasmid containing a polynucleotide encoding the CAR. Twenty-four hours post-transfection, cells are replenished with a basal culture medium supplemented with 10% FBS and incubated at 5% CO₂ and 37 degrees Celsius. Forty-eight hours later, the first harvest of viral vectors is performed and the producer cells are replenished with the medium mentioned above. Another 24 hours later, the second, and final, harvest is performed.
Next, both harvests are combined, filtered through a 0.22 um or 0.45 um PES filter and treated with benzonase (50 U/ml) to remove any of the remaining plasmid DNA. Next, viral supernatant is concentrated by PEG6000 precipitation. Briefly, viral supernatant is mixed with 4× Precipitation Buffer (consisting of 0.5M NaCl and 7.5% PEG6000) and centrifuged for 20 minutes at 7000×g at 4 degrees Celsius. Next, the supernatant is removed and resuspended in the appropriate culture medium and used immediately for downstream transduction or stored at −80 degrees Celsius for later use.
Here, we distinguish 4 different growth media:

| | Basal Medium | Additional cytokines |
|---|---|---|
| MEDIUM A | CellGenix ® GMP | 100 ng/ml Flt3-L, 100 ng/ml |
| | Stem cell Growth | SCF, 50 ng/ml TPO |
| | Medium | and 50 ng/ml IL-3 |
| MEDIUM B | CellGenix ® GMP | 100 ng/ml Flt3-L, 100 ng/ml |
| | Stem cell Growth | SCF, 50 ng/ml TPO |
| | Medium | and 50 ng/ml IL-6 |
| MEDIUM C | Glycostem Basal | 100 ng/ml Flt3-L, 100 ng/ml |
| | Growth Medium | SCF, 50 ng/ml TPO |
| | | and 50 ng/ml IL-7 |
| MEDIUM D | Glycostem Basal | 25 ng/ml Flt3-L, 25 ng/ml |
| | Growth Medium | SCF, 25 ng/ml TPO and 25 ng/ml |
| | | IL-7, 10% human serum, 10 pg/ml |
| | | GM-CSF, 250 pg/ml G-CSF |
| | | and 50 pg/ml IL6 |

### Example 2: Transduction of CD34⁺ Hematopoietic Stem Cells by Lentiviral Vector

During the first phase of the method of the invention, CD34+ stem cells are isolated from starting material by immunomagnetic separation technique and subsequently transduced by lentiviral vector containing one or more polynucleotides encoding a CAR, yielding CAR-NK cells. The goal is to obtain a stable population of CAR-NK cells that are expanded in the second phase of the method of the invention.

### Starting Material

The starting material to be used in the method of the present invention is a biological sample containing adult (postembryonic) stem cells, also called somatic stem cells. As used herein, the term biological sample means a sample derived from a human being. In a preferred embodiment, the starting material to be used is umbilical cord blood.

### Isolation of CD34+ Stem Cell Population

CD34+ stem cells are isolated from umbilical cord blood via the Miltenyi Prodigy® closed-system cell manufacturing platform. Briefly, cells are purified by magnetic separation via CD34-conjugated magnetic beads. The end product is a pure CD34+ population that is collected in culture medium I and is either used for expansion and/or differentiation, transduction or frozen down in a freezer via a controlled temperature profile.

### Transduction of CD34+ Cells to Obtain a CAR-NK Population

Immediately following isolation or thawing, CD34+ stem cells are counted and immediately resuspended in the viral stock prepared in example 1 at an MOI of 1-50 and plated in Retronectin-coated plates. Optionally, transduction efficiency may be enhanced by the addition of 10 ug/ml polybrene or 10 ug/ml protamine sulfate.
Including control cells, we distinguish:

| Cells | Medium | MOI | Condition |
|---|---|---|---|
| CD34+ | MEDIUM A | 1 | CAR-T vector containing scFV against BCMA |
| CD34+ | MEDIUM A | 10 | CAR-T vector containing scFV against BCMA |
| CD34+ | MEDIUM A | 50 | CAR-T vector containing scFV against BCMA |
| CD34+ | MEDIUM A | 1 | CAR-T vector containing scFv against EGFR |
| CD34+ | MEDIUM A | 10 | CAR-T vector containing scFv against EGFR |
| CD34+ | MEDIUM A | 50 | CAR-T vector containing scFv against EGFR |
| CD34+ | MEDIUM A | 1 | CAR-T vector containing irrelevant scFV (CAR-NK-IRR) |
| CD34+ | MEDIUM A | 10 | CAR-T vector containing irrelevant scFV (CAR-NK-IRR) |
| CD34+ | MEDIUM A | 50 | CAR-T vector containing irrelevant scFV (CAR-NK-IRR) |
| CD34+ | MEDIUM A | N/A | MOCK |
| CD34+ | MEDIUM B | 1 | CAR-T vector containing scFV against BCMA |
| CD34+ | MEDIUM B | 10 | CAR-T vector containing scFV against BCMA |
| CD34+ | MEDIUM B | 50 | CAR-T vector containing scFV against BCMA |
| CD34+ | MEDIUM B | 1 | CAR-T vector containing scFv against EGFR |
| CD34+ | MEDIUM B | 10 | CAR-T vector containing scFv against EGFR |
| CD34+ | MEDIUM B | 50 | CAR-T vector containing scFv against EGFR |
| CD34+ | MEDIUM B | 1 | CAR-T vector containing irrelevant scFV (CAR-NK-IRR) |
| CD34+ | MEDIUM B | 10 | CAR-T vector containing irrelevant scFV (CAR-NK-IRR) |
| CD34+ | MEDIUM B | 50 | CAR-T vector containing irrelevant scFV (CAR-NK-IRR) |
| CD34+ | MEDIUM B | N/A | MOCK |
| CD34+ | MEDIUM C | 1 | CAR-T vector containing scFV against BCMA |
| CD34+ | MEDIUM C | 10 | CAR-T vector containing scFV against BCMA |
| CD34+ | MEDIUM C | 50 | CAR-T vector containing scFV against BCMA |
| CD34+ | MEDIUM C | 1 | CAR-T vector containing scFv against EGFR |
| CD34+ | MEDIUM C | 10 | CAR-T vector containing scFv against EGFR |
| CD34+ | MEDIUM C | 50 | CAR-T vector containing scFv against EGFR |
| CD34+ | MEDIUM C | 1 | CAR-T vector containing irrelevant scFV (CAR-NK-IRR) |
| CD34+ | MEDIUM C | 10 | CAR-T vector containing irrelevant scFV (CAR-NK-IRR) |
| CD34+ | MEDIUM C | 50 | CAR-T vector containing irrelevant scFV (CAR-NK-IRR) |
| CD34+ | MEDIUM C | N/A | MOCK |
| CD34+ | MEDIUM D | 1 | CAR-T vector containing scFV against BCMA |
| CD34+ | MEDIUM D | 10 | CAR-T vector containing scFV against BCMA |
| CD34+ | MEDIUM D | 50 | CAR-T vector containing scFV against BCMA |
| CD34+ | MEDIUM D | 1 | CAR-T vector containing scFv against EGFR |
| CD34+ | MEDIUM D | 10 | CAR-T vector containing scFv against EGFR |
| CD34+ | MEDIUM D | 50 | CAR-T vector containing scFv against EGFR |
| CD34+ | MEDIUM D | 1 | CAR-T vector containing irrelevant scFV (CAR-NK-IRR) |
| CD34+ | MEDIUM D | 10 | CAR-T vector containing irrelevant scFV (CAR-NK-IRR) |
| CD34+ | MEDIUM D | 50 | CAR-T vector containing irrelevant scFV (CAR-NK-IRR) |
| CD34+ | MEDIUM D | N/A | MOCK |

Optionally, 24 hours post-transduction, cells are washed with the selected medium and are re-transduced at the same MOI. Following 24 hours of incubation, cells are replenished with the culture medium II.

### Example 3: Expansion and Differentiation of CAR-CD34+ Cells

### Cell Culture

The transduced cells of condition A-D are cultured directly, or if cryopreserved CAR transduced and MOCK progenitor cells from condition A-D are thawed in thawing buffer consisting of human serum albumin supplemented with 2.5 mM MgCl2 and 0.13 mg/ml DNAse. The CD34+ UCB cells are plated in tissue culture treated 6-wells plates in fresh culture medium I at cell concentrations of 0.02-2×10\hat{ }6 cells/ml for conditions A-D.

### Expansion Phase

Flow cytometry data for cell viability, CD34 content and CAR expression are measured in the expansion phase to monitor and provide optimal cell culture conditions. CD34+ UCB cells are cultured in fresh culture medium I consist of GBGM supplemented with 10% human serum, a low dose cytokine cocktail containing 10 pg/ml GM-CSF, 250 pg/ml G-CSF and 50 pg/ml IL-6; 25 ng/ml SCF, Flt-3L, TPO, IL-7. Cells from condition A-D are cultured in culture medium I till day 9. Culture medium I is refreshed every 2-3 days a week. At day 10, the progenitor cells are cultured by adding culture medium II, hereby replacing 25 mg/ml TPO for 20 ng/ml IL-15.

### Differentiation Phase

During differentiation, the CD56 content as a marker of NK-cells is measured instead of the CD34 content alongside cell viability and CAR expression. Differentiation medium (aka culture medium III) consists of GBGM supplemented with 2% human serum, a low dose cytokine cocktail containing 10 pg/ml GM-CSF, 250 pg/ml G-CSF and 50 pg/ml IL-6; 20 ng/ml SCF, IL-15, IL-7 and 1000 U/ml IL-2 (Proleukin) Differentiation medium is refreshed twice a week until end of culture.
The CAR expression in the BCMA and EGFR transduced UCB-NK cells reveals relative high CAR expression in condition A-C transduced NK-cells and mediate expression in condition D transduced NK-cells.

### Example 4: Anti-Tumor Potential of CAR-NK-BCMA and CAR-NK-EGFR Cells Versus Control CAR-NK-IRR and MOCK Cells

An in vitro functionality assay was performed at the end of the culture with Mock and CAR transduced UCB-NK cells from condition A-D against a battery of multiple myeloma and colorectal cancer cell lines (see table below) showing sensitive-, intermediate- and resistance to NK-cell cytotoxicity.

| Myeloma | Colorectal Cancer |
|---|---|
| Cell lines | Cell lines |
| RPMI-8226 | HT-29 |
| U-266 | TC71 |
| OPM-2 | HCT-15 |
| LP-1 | SW480 |
| SK-MM2 | COLO320 |
| NCI-H929 | RKO |

Target cells (see table above) were labeled with 5 μM pacific blue succinimidyl ester (PBSE) at a concentration of 1×10\hat{ }7 cells/ml for 10 minutes at 37° C. The target cells were washed in target culture medium and concentrated to 5×10\hat{ }5 cells/ml. The NK-cells were concentrated to 5×10\hat{ }5 cells/ml as well and co-cultured with target cells (100 μl effectors+100 μl targets) in an overnight assay. For degranulation measurements anti-CD107a was added at the start of the incubation and anti-CD56 for NK-cell discrimination at the end of the incubation. Cytotoxicity was calculated based on flow cytometry read out for the apoptotic 7AAD viability marker for Effector:Target (E:T) ratios of 1:1.
Lower cytotoxicity is observed for the BCMA and EGFR transduced UCB-NK cells from condition A-C against the sensitive tumour cell lines (see table above) compared to condition D.

### Example 5: Expansion and Differentiation of CD34+ Cells

### Cell Culture

CD34+ UCB cells are plated in tissue culture treated 6-wells plates in fresh culture medium I at cell concentrations of 0.02-2×10\hat{ }6 cells/ml for conditions A-D.

### Expansion Phase

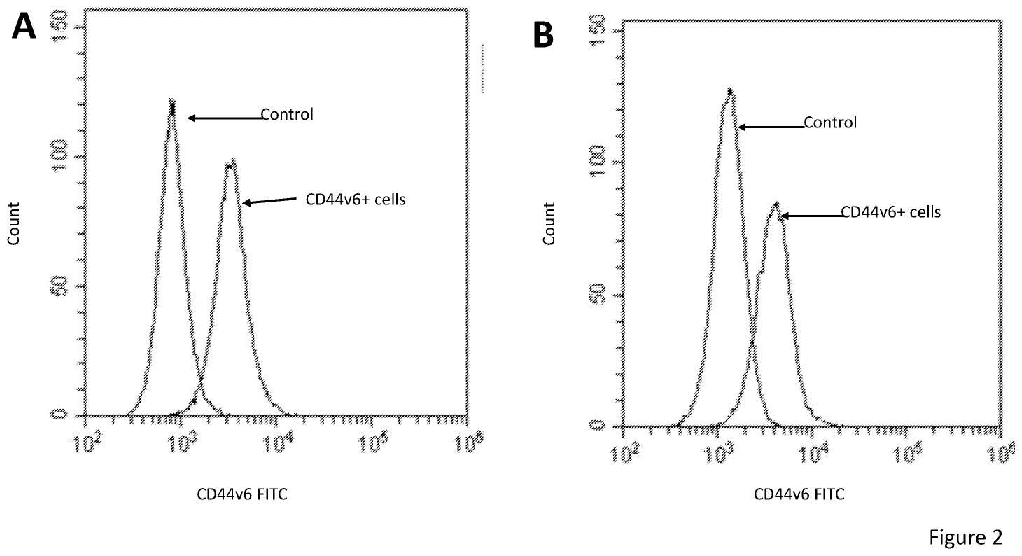

Flow cytometry data for CD45 and CD56 as well as CD44v6 are measured in the expansion phase to monitor and provide optimal cell culture conditions. CD34+ UCB cells are cultured in fresh culture medium I consist of GBGM supplemented with 10% human serum, a low dose cytokine cocktail containing 10 pg/ml GM-CSF, 250 pg/ml G-CSF and 50 pg/ml IL-6; 25 ng/ml SCF, Flt-3L, TPO, IL-7. Cells are cultured in culture medium I till day 9. Culture medium I is refreshed every 2-3 days a week. At day 10, the progenitor cells are cultured by adding culture medium II, hereby replacing 25 mg/ml TPO for 20 ng/ml IL-15. At day 14 cell culture is analysed for CD56 NK cell content and CD44v6 expression as shown in FIG. 2.

### Differentiation Phase

During differentiation, the CD56 content as a marker of NK-cells is measured together with CD45 and CD44v6 expression. Differentiation medium (aka culture medium III) consists of GBGM supplemented with 2% human serum, a low dose cytokine cocktail containing 10 pg/ml GM-CSF, 250 pg/ml G-CSF and 50 pg/ml IL-6; 20 ng/ml SCF, IL-15, IL-7 and 1000 U/ml IL-2 (Proleukin). Differentiation medium is refreshed twice a week until end of culture. At day 29 cell culture is analysed for CD56 NK cell content and CD44v6 expression as shown in FIG. 2.

### Example 6: Transduction of CD34⁺ Hematopoietic Stem Cells and Precursor Cells Derived from CD34+ Hematopoietic Stem Cells by Lentiviral Vector

CD34+ stem cells are isolated from starting material by immunomagnetic separation technique and subsequently transduced with a lentiviral vector, pseudotyped by a VSVG envelop, containing one or more polynucleotides encoding an EGFP, yielding EGFP-NK cells (pLenti CMV GFP Puro (658-5); Addgene plasmid #17448; n2t.net/addgene:17448; RRID:Addgene_17448; Campeau E, Ruhl V E, Rodier F, Smith C L, Rahmberg B L, Fuss J O, Campisi J, Yaswen P, Cooper P K, Kaufman P D. PLoS One. 2009 Aug. 6; 4(8):e6529). Alternatively, CD34+ stem cells are cultured as described previously (Method III in Spanholtz J, Tordoir M, Eissens D, Preijers F, van der Meer A, Joosten I, et al. PLoS ONE 2010 5(2): e9221) to achieve precursor cells; and these precursor cells are transduced with the above lentiviral vector at day 21, 28, or 34, before further expansion and differentiation.

### Starting Material

Here, we distinguish 2 different growth media:

| | Basal Medium | Additional cytokines |
|---|---|---|
| MEDIUM A | Glycostem Basal | 50 ng/ml Flt3-L, 50 ng/ml |
| | Growth Medium | SCF, 50 ng/ml TPO 50 ng/ml |
| | | IL-7, 20 pg/ml GM-CSF, 500 pg/ml |
| | | G-CSF and 100 pg/ml IL6 |
| MEDIUM B | Glycostem Basal | 20 ng/ml SCF, 20 ng/ml |
| | Growth Medium | IL-7, 2% human serum, 10 pg/ml |
| | | GM-CSF, 250 pg/ml G-CSF, 50 |
| | | pg/ml IL6, 25 ng/mL IL-7, 20 ng/mL |
| | | IL-15, 20 ng/mL SCF, 1000 U/mL IL-2 |

### Isolation of CD34+ Stem Cell Population

CD34+ stem cells are isolated from umbilical cord blood via the Miltenyi Prodigy® closed-system cell manufacturing platform (Miltenyi Biotec B.V.&Co KG, Bergisch Gladbach, Germany). Briefly, cells are purified by magnetic separation via CD34-conjugated magnetic beads. The end product is a pure CD34+ population that is collected in culture medium I and is either used for expansion and/or differentiation, or frozen down in a freezer via a controlled temperature profile.

### Transduction of CD34+ Cells or Precursors Derived Thereof to Obtain an EGFP-NK Population

Immediately following thawing, CD34+ stem cells resuspended in Medium A. Twenty-four hours later cells are counted, resuspended at a concentration of 5,000 cells per 100 μl of fresh Medium A per well in 96-well plates. Viral stock prepared in example 1 was diluted in Medium A at an MOI of 20 per 100 μl in Retronectin-coated plates (Takara Bio Europe SAS, St Germain en Laye, France). Cell were added to the plates subsequently. Optionally, transduction efficiency may be enhanced by the addition of following transduction enhancers to the viral stock to reach these final concentrations in Medium A: 8 μg/ml polybrene (Sigma-Aldrich Chemie N.V., Zwijndrecht, The Netherlands), 4 μ/ml protamine sulfate (Sigma-Aldrich Chemie N.V.), 1 μg/ml Vectofusin®-1 (Miltenyi Biotec B.V.&Co KG), 4 μ/ml protamine sulfate plus 1 μg/ml Vectofusin®-1, 1 mg/ml LentiBOOST™ (Sirion Biotech GmbH, Martinsried, Germany), or 1 μl/100 μl media of LentiBlast Premium (OZBiosciences SAS, Marseille, France).
Alternatively, CD34+ cells are cultured in expansion and differentiation medium and resulting precursor cells are transduced as described above. Transduction was performed with cells resuspended at a concentration of 200,000 cells per 250 μl of fresh Medium B per well in 24-well plates. Viral stock prepared in example 1 was diluted in Medium B at an MOI of 20 per 250 μl. Transduction enhancers were used at the same final concentrations as described above.
Including control cells, we distinguish:

| Cells | Medium | MOI | Condition |
|---|---|---|---|
| CD34+ | MEDIUM A | 20 | EGFP vector |
| CD34+ | MEDIUM A | N/A | MOCK |
| Precursors derived from CD34+ cells at day 21 | MEDIUM B | 20 | EGFP vector |
| Precursors derived from CD34+ cells at day 21 | MEDIUM B | N/A | MOCK |
| Precursors derived from CD34+ cells at day 28 | MEDIUM B | 20 | EGFP vector |
| Precursors derived from CD34+ cells at day 28 | MEDIUM B | N/A | MOCK |
| Precursors derived from CD34+ cells at day 35 | MEDIUM B | 20 | EGFP vector |
| Precursors derived from CD34+ cells at day 35 | MEDIUM B | N/A | MOCK |

Following 24 hours of incubation, CD34+ cells are replenished with the culture medium II. Following 24 hours of incubation, precursors derived from CD34+ cells are replenished with the culture medium III.

1-16. (canceled)

17. A method for the manufacturing of a population of cells, genetically modified with a Chimeric Antigen Receptor (CAR) comprising:
a first step comprising:
providing a sample comprising CD34+ hematopoietic stem cells
purifying the CD34+ hematopoietic stem cells in said sample,
culturing the purified CD34+ hematopoietic stem cells in the presence of culture medium I,
transducing the purified CD34+ hematopoietic stem cells with a polynucleotide coding for a CAR by culturing the cellular populations in culture medium I in the presence of a vector comprising said polynucleotide for at least 10 hours, thereby obtaining a cellular population comprising CD34+ stem cells expressing said CAR, and
culturing the cellular populations in culture medium I for at least 10 hours, wherein
culture medium I is a basic culture medium, comprising a collection of cytokines, wherein said collection of cytokines comprises Interleukin-7 and one or more of stem cell factor (SCF), flt-3Ligand (FLT-3L), thrombopoietin (TPO), and two or more of granulocyte-macrophage-colony-stimulating factor (GM-CSF), granulocyte-colony-stimulating factor (G-CSF), and interleukin-6 (IL-6).

18. The method according to claim 17, wherein in step d) the cellular populations in culture medium I are cultured in the presence of a vector comprising said polynucleotide for at least 16 hours.

19. The method according to claim 17, wherein in step e) the cellular populations in culture medium I are cultured for at least 48 hours.

20. The method according to claim 17, wherein the cell culture of step c) is initiated at a cell density of between 500 and 10,000 CD34⁺ cells/ml.

21. The method according to claim 20, wherein the cell culture of step c) is initiated at a cell density of between 1,000 and 8,000 CD34+ cells/ml.

22. The method according to claim 17, wherein the polynucleotide does not encode for a CAR specific for an antigen expressed on hematopoietic stem cells, natural killer (NK) progenitor cells or NK cells.

23. The method according to claim 17, further comprising:
a second step in which the cellular population from step (i) is expanded and differentiated into a cellular population containing CAR-NK progenitor cells or CAR-NK cells or both, the step comprising culturing the cellular population from step (i) containing CAR-CD34+ stem cells in culture medium III, thereby obtaining a cellular population containing CAR-NK progenitor cells or CAR-NK cells or both, wherein the culture medium III is a basic culture medium comprising a collection of cytokines, wherein said collection of cytokines comprises two or more of SCF, IL-7, IL-15 and interleukin-2 (IL-2) and two or more of GM-CSF, G-CSF, and IL-6.

24. The method according to claim 17, further comprising:
a second step in which the cellular population from step (i) is expanded and differentiated into a cellular population containing CAR stem cells or CAR progenitor cells or both, the step comprising culturing the CAR-CD34+ stem cells from step (i) in culture medium II, thereby obtaining a cellular population containing CAR stem cells or CAR progenitor cells or both, and
a third step in which the cellular population from step (ii) is further expanded and differentiated into a cellular population containing CAR-NK progenitor cells or CAR-NK cells or both,
wherein culture medium II is a basic culture medium comprising a collection of cytokines, wherein said collection of cytokines comprises two or more of SCF, FLT-3L interleukin-15 (IL-15) and IL-7 and two or more of GM-CSF, G-CSF, and IL6, and wherein culture medium III is a basic culture medium comprising a collection of cytokines, wherein said collection of cytokines comprises two or more of SCF, IL-7, IL-15 and interleukin-2 (IL-2) and two or more of GM-CSF, G-CSF, and IL-6.

25. The method according to claim 17, wherein the sample has been obtained from umbilical cord blood.

26. The method according to claim 17, wherein stem cells are purified using CD34+ immunomagnetic selection methods.

27. The method according to claim 17, wherein culture medium I comprises SCF at concentration between 4 ng/ml and 300 ng/ml, or Flt3-L at concentration between 4 ng/ml and 300 ng/ml, or TPO at concentration between 4 ng/ml and 100 ng/ml, or IL7 at concentration between 4 ng/ml and 50 ng/ml, or any combination of these cytokines in the specified concentrations.

28. The method according to claim 17, wherein CD34+ stem cells are incubated with a viral vector containing said polynucleotide coding for said CAR at multiplicity of infection (MOI) between 0.01 and 100

29. The method according to claim 28, wherein CD34+ stem cells are incubated with a viral vector containing said polynucleotide coding for said CAR at multiplicity of infection (MOI) between 1 and 10.

30. The method according to claim 17, wherein the viral vector is a retroviral vector.

31. The method according to claim 17, wherein the CAR is directed against a tumour antigen.

32. A composition comprising an NK-CAR cell obtainable by a method according to claim 17.

33. The composition according to claim 32, wherein the NK-CAR cell is positive for Neural Cell Adhesion Molecule (NCAM) and a CAR.

34. A method for treating a subject having a tumour or a haematological malignancy, comprising administering to the subject a composition according to claim 32.


SEQUENCE LISTING (11 sequences)

SEQ ID NO: 1 - protein, 239 aa, Homo sapiens
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 2 - protein, 242 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 3 - protein, 26 aa, Artificial Sequence
  Part of CAR cassete - CD8a leader sequence
MDALPVTALLLPLALLLHAEVQLQQS

SEQ ID NO: 4 - protein, 11 aa, Artificial Sequence
  Part of CAR casette - FseI-SbfI flanked stuffer fragment
GRPSLSESCRA

SEQ ID NO: 5 - protein, 26 aa, Artificial Sequence
  Part of CAR cassette - IgD hinge region
AFRWPESPKAQASSVPTAQPQAEGSL

SEQ ID NO: 6 - protein, 274 aa, Artificial Sequence
  Part of CAR cassette - Optimized IgG1 heavy chain Fc part, optimized
AKATTAPATTRNTGGRGGEEKKKEKEKEEQEERETKTPEAAAVEPKSCDKTHTCPPCAAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 27 aa, Artificial Sequence
  Part of CAR cassette - CD28 transmembrane domain
FWVLVVVGGVLACYSLLVTVAFIIFWV

SEQ ID NO: 8 - protein, 41 aa, Artificial Sequence
  Part of CAR cassette - CD28 co-activation domain
RSKRSRLLHSDYMNMTPRRPGPTRKHYQPYAPPRDFAAYRS

SEQ ID NO: 9 - protein, 37 aa, Artificial Sequence
  Part of CAR cassette - OX40 co-activation domain
RRDQRLPPDAHKPPGGGSFRTPIQEEQADAHSTLAKI

SEQ ID NO: 10 - protein, 112 aa, Artificial Sequence
  Part of CAR cassette - CD3zeta activation domain
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 8619 bp, Artificial Sequence
  pLenti CMV GFP Puro (658-5)
aggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatctcgagggcccggtacctttaagaccaatgacttacaaggcagctgtagatcttagccactttttaaaagaaaaggggggactggaagggctagctcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaattt